(19) United States
(12) Patent Application Publication
(10) Pub. No.: US 2003/0152513 A1
(43) Pub. Date: Aug. 14, 2003

(54) Intravascular delivery of therapeutic and imaging agents to stressed and apoptotic cells using annexin V as a targeting vector

(75) Inventors: Francis G. Blankenberg, Menlo Park, CA (US); Jagat Narula, Seattle, WA (US); H. William Strauss, Redwood City, CA (US); Vartan Ghazarossian, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IMETRIX, INC., Mountain View, CA

(21) Appl. No.: 10/238,026

(22) Filed: Sep. 6, 2002

Related U.S. Application Data
(60) Provisional application No. 60/318,171, filed on Sep. 6, 2001.

(51) Int. Cl. A61K 51/00

(52) U.S. Cl. 424/1.49; 530/391.1; 424/1.69

(57) ABSTRACT

The present invention relies on the affinity of stressed or apoptotic cells for exogenously administered annexin V to create a multi-functional molecular probe that can be simultaneously used for imaging (localization of unstable plaque within the body) and therapy (treatment of unstable plaque).

### CROSS-REFERENCES TO RELATED APPLICATIONS

This application claims the benefit of prior provisional application No. 60/318,171 (Attorney Docket No. 020039-002100), filed on Sep. 6, 2001, under 37 CFR §1.78(a)(3), the full disclosure of which is incorporated herein by reference.

### BACKGROUND OF THE INVENTION

1. Field of the Invention

The present invention relates generally to medical devices and methods. More particularly, the present invention relates to nuclear radiology and devices and methods for the intraluminal characterization and/or treatment of lesions in blood vessels and other body lumens.

Coronary artery disease resulting from the build-up of atherosclerotic plaque in the coronary arteries is a leading cause of death in the United States and worldwide. The plaque build-up causes a narrowing of the artery, commonly referred to as a lesion, which reduces blood flow to the myocardium (heart muscle tissue). Myocardial infarction (better known as a heart attack) can occur when an arterial lesion abruptly closes the vessel, causing complete cessation of blood flow to portions of the myocardium. Even if abrupt closure does not occur, blood flow may decrease resulting in chronically insufficient blood flow which can cause significant tissue damage over time.

A variety of interventions have been proposed to treat coronary artery disease. For disseminated disease, the most effective treatment is usually coronary artery bypass grafting where problematic lesions in the coronary arteries are bypassed using external grafts. In cases of less severe disease, pharmaceutical treatment is often sufficient. Finally, focal disease can often be treated intravascularly using a variety of catheter-based approaches, such as balloon angioplasty, atherectomy, radiation treatment, stenting, and often combinations of these approaches.

With the variety of treatment techniques which are available, the cardiologist is faced with a challenge of selecting the particular treatment which is best suited for an individual patient. While numerous of diagnostic aids have been developed, no one technique provides all the information which is needed to select a treatment. Angiography is very effective in locating lesions in the coronary vasculature, but provides little information concerning the nature of the lesion. To provide better characterization of the lesion(s), a variety of imaging techniques have been developed for providing a more detailed view of the lesion, including intravascular ultrasound (IVUS), angioscopy, laser spectroscopy, computed tomography (CT), magnetic resonance imaging (MRI), and the like. None of these techniques, however, is completely successful in determining the exact nature of the lesion. In particular, such techniques provide little information regarding whether the plaque is stable or unstable.

Plaques which form in the coronaries and other vessels comprise inflammatory cells, smooth muscles cells, cholesterol, and fatty substances, and these materials are usually trapped between the endothelium of the vessel and the underlying smooth muscle cells. Depending on various factors, including thickness, composition, and size of the deposited materials, the plaques can be characterized as stable or unstable. The plaque is normally covered by an endothelial layer. When the endothelial layer is disrupted, the ruptured plaque releases highly thrombogenic constituent materials which are capable of activating the clotting cascade and inducing rapid and substantial coronary thrombosis. Such rupture of an unstable plaque and the resulting thrombus formation can cause unstable angina chest pain, acute myocardial infarction (heart attack), sudden coronary death, and stroke. It has recently been proposed that plaque instability, rather than the degree of plaque build-up, should be the primary determining factor for treatment selection.

A variety of approaches for distinguishing stable and unstable plaque in patients have been proposed. Some of the proposals involve detecting a slightly elevated temperature within unstable plaque resulting from inflammation. Other techniques involve exposure of the plaque to infrared light. It has also been proposed to introduce radiolabeled materials which have been shown by autoradiography to bind to stable and unstable plaque in different ways. External detection of the radiolabels, however, greatly limits the sensitivity of these techniques and makes it difficult to determine the precise locations of the affected regions. Thus far, none of these technologies has possessed sufficient sensitivity or resolution necessary to reliably characterize the plaque at the cellular level in the intact animal or man.

In pending application Ser. No. 09/670,412 filed on Sep. 26, 2000, the inventor herein proposes the in situ detection of labeled markers within body lumens to provide information on proliferative conditions within the lumens. In particular, the use of radiolabeled binding substances, such as low-density lipoproteins, cellular precursors, including proteins, nucleic acids, and the like were proposed to provide for targeted binding at the proliferative sites. Specific binding substances listed in the application were monocyte chemoattractant peptide 1 (MCP1), Z2D3 antibody, and fluorodeoxyglucose.

Our research has shown that both technetium-99m hydrazino nicontiamide and biotin labeled annexin V can localize in vivo following intravenous injection to neurons, astrocytes, cardiomyocytes in regions of reversible and irreversible ischemic reperfusion injury (Blankenberg, 2000 and Narula, 2000). These experiments have also clearly demonstrated that annexin V along with its label can cross both the cell membrane and the blood brain barrier and selectively localize to cells that are either physiologically stressed, or that are apoptotic.

Other studies have shown that annexin V itself has anti-apoptotic effects in vivo (Gidon-Jeangirard C, 1999) in addition to its inhibitory effects on membrane permeability to calcium, protein kinase C and phospholipase A₂ in vitro (Gidon-Jeangirard C, 1999 and Russo-Marie F, 1999).

The localization of annexin V in vivo is dependent on the selective exposure of phosphatidylserine (PS), a ubiquitous membrane bound anionic phospholipid, on the surface of stressed or apoptotic cells. Normally PS is actively restricted to the inner leaflet of the plasma membrane by translocase, an anionic ATP-dependent aminophospholipid pump which serves to preserve the normal plasma cell membrane asymmetry in mammalian cells (Zwaal 1997). PS is selectively exposed on the surfaces of cells that are severely stressed or apoptotic. The exposure of PS on the cell surface serves as a marker for adjacent healthy cells to phagocytose apoptotic cells and their remnants (Fadok V A, 2000).

Annexin V binds to the surface of stressed and apoptotic cells in the presence of physiologic levels of extracellular calcium with a high affinity (i.e. 1-10 nmol/L). Annexin V can also enter cells by an unknown mechanism. Possibilities include entry via pinocytosis, via other endocytic mechanisms, or by an as yet unidentified pump mechanism specific to annexins or annexin V.

The exposure of PS on the cell surface also precedes the commitment to apoptotic cell death and can therefore be reversible in cells when the signal-induced apoptotic stress is removed or inhibited in a timely fashion permitting continued cell viability and the resumption of normal cell function and growth (Hammill A K, 1999). This observation suggests that annexin V can not only be used to target apoptotic cells but also those cells which though severely injured maybe capable of recovery or of being salvaged through therapeutic intervention (Strauss H W, 2000).

For all of these reasons, it would be desirable to provide improved methods and apparatus which are capable of both distinguishing between stable and unstable plaque within the coronary and other patient vasculature as well as treating the plaque which has been identified as being unstable to enhance stability. At least some of these objectives will be met by the inventions described hereinafter.

2. Description of the Background Art

U.S. Pat. Nos. 6,197,278; 6,171,577 and 5,968,477 described the preparation of radiolabeled annexins and their use for imaging thrombus in the vasculature. Stratton et al. (1995) Circulation 92:3113-3121, considers the use of radiolabeled annexin V for intra-arterial thrombus detection. The use of radiolabeled agents for detecting atherosclerotic lesions is described in the medical literature. See, for example, Elmaleh et al. (1998) Proc. Natl. Acad. Sci. USA 95:691-695; Vallabhajosula and Fuster (1997) J. Nucl. Med. 38:1788-1796); Demos et al. (1997) J. Pharm. Sci. 86:167-171; Narula et al. (1995) Circulation 92: 474-484; and Lees et al. (1998) Arteriosclerosis 8:461470. U.S. Pat. No. 4,660,563, describes the injection of radiolabeled lipoproteins into a patient where the lipoproteins are taken up into regions of arteriosclerotic lesions to permit early detection of those lesions using an external scintillation counter. U.S. Pat. No. 5,811,814, describes and intravascular radiation-detecting catheter. The catheter is used to locate tagged red blood cells that may accumulate, for example, in an aneurysm. U.S. Pat. No. 5,429,133, describes a laparoscopic probe for detecting radiation concentrated in solid tissue tumors. Miniature and flexible radiation detectors intended for medical use are produced by Intra-Medical LLC, Santa Monica, Calif. (www.intra-medical.com). See also U.S. Pat. Nos. 4,647,445; 4,877,599; 4,937,067; 5,510,466; 5,711,931; 5,726,153; and WO 89/10760.

The following publications some of which are referenced above are also pertinent:

1. D'Arceuil H, et al. 99m Tc annexin V imaging of neonatal hypoxic brain injury. Stroke 2000; 31:71-75.

2. Narula J, et al. Transient sarcolemmal phosphatidylserine expression as a marker of brief ischemia: An evaluation by 99m Tc-annexin V imaging. Journal of Nuclear Medicine 2000; 41:Suppl. p.173-174P.

3. Gidon-Jeangirard C, et al. Annexin V delays apoptosis while exerting an external constraint preventing the release of CD4+ and PrPc+ membrane particles in a human T lymphocyte model. Journal of Immunology 1999; 162:5712-5718.

4. Gidon-Jeangirard C, et al. Annexin V counteracts apoptosis while inducing Ca(2+) influx in human lymphocytic cells. Biochem Biophys Res Commun. 1999; 265:709-715.

5. Russo-Marie F. Annexin V and phospholipid metabolism. Clin Chem Lab Med 1999; 37:287-291.

6. Zwaal R F A, Schroit A J. Pathophysiologic implications of membrane phospholipid asymmetry in blood cells. Blood 1997; 89:1121-1132.

7. Fadok V A, et al. A receptor for phosphatidylserine specific clearance of apoptotic cells. Nature 2000; 405:85-90.

8. Hammill A K, et al. Annexin V staining due to loss of membrane symmetry can be reversible and precede commitment to apoptotic death. Exp Cell Res. 1999; 251:16-21.

9. Strauss H W, et al. Radioimaging to identify myocardial death and probably injury. Lancet 2000; 356:180.

### BRIEF SUMMARY OF THE INVENTION

The present invention relies on the affinity of stressed or apoptotic cells for exogenously administered annexin V to create a multi-functional molecular probe that can be simultaneously used for imaging (localization of unstable plaque within the body) and therapy (treatment of unstable plaque).

In a first embodiment, annexin V is labeled with both a radioisotope such as technetium-99m and a photodynamic agent such as a light absorbing porphyrin. After intravenous or intra-arterial injection of the bifunctional annexin V complex, lesions of interest such as vulnerable (apoptotic) atherosclerotic plaques would be located with an endovascular scintillation detector that would preferably have a laser or other source that would emit light of a wavelength matching the absorption wavelength of the porphyrin. Targeted cells sensitized to light through the localization of the annexin V complex are then selectively destroyed with a limited laser pulse, minimizing damage to adjacent healthy cells and tissue.

Conversely, annexin V could be conjugated with antisense-DNA or RNA oligonucleotides with a label bond that would lyse upon entry into the target cell trapping the oligonucleotide(s) of interest within. Radiolabeling would also permit the noninvasive detection of the localization of annexin V conjugates in vivo. Other therapeutic motifs could also be employed.

The intrinsic anti-apoptotic properties of internalized annexin V could also be exploited whereby radiolabeled annexin V for imaging could be co-injected with much greater amounts of unlabeled annexin V for therapeutic effect. In addition large saturating quantities of annexin V may also have an in vivo anti-inflammatory effect by blocking PS recognition by macrophages and lymphocytes.

In particular, compositions according to the present invention for detecting and treating vulnerable plaque comprise a binding molecule, a targeting molecule, and an effector molecule. The binding molecule will specifically bind to marker(s) on stressed or apoptotic cells which are characteristic of vulnerable plaque. The targeting molecule will permit localization of the composition when the composition is intravascularly bound to vulnerable plaque. Finally, the effector molecule will selectively kill or inhibit the stressed or apoptotic cells associated with vulnerable plaque. In a first specific embodiment, the binding molecule comprises annexin. In a second specific embodiment, the targeting molecule comprises a radiolabel such as technetium-99m. In a third specific embodiment, the effector molecule comprises a photodynamic agent such as a porphyrin.

In an alternative aspect of the present invention, the compositions may comprise or consist essentially of an annexin, such annexin VI, coupled or otherwise bound to a targeting molecule, such as a radiolabel such as technetium-99m. The annexin is believed to both provide binding and provide a therapeutic benefit when bound to the apoptotic or stressed cells characteristic of vulnerable plaque. The annexin compositions, of course, may be further bound to a porphyrin or other photodynamic or other effector molecule, generally as described above.

Methods according to the present invention for detecting and treating vulnerable plaque comprise administering a composition to a patient suspected of having vulnerable plaque. The composition is capable of specifically binding to the vulnerable plaque, being localized when bound (i.e., detected), and killing or inhibiting the apoptotic or stressed cells characteristic of vulnerable plaque. The methods further comprise determining whether the composition has localized. If the composition has localized, the plaque is determined to be unstable and the patient will be diagnosed as suffering from vulnerable plaque. The treating physician will then activate the composition to kill or inhibit the apoptotic or stressed cells. Usually, the composition will comprise an effector molecule, such as a photodynamic agent such as porphyrin, as described above. Activation will then comprise exposing the localized composition to light in order to activate the photodynamic agent.

Preferably, both detection and activation may be achieved using an intravascular catheter having components adapted to both detect the label, e.g. a radio nuclide or other detector, as well as to activate the photodynamic agent, e.g. a light source such as a fiber optic tube, an LED, a scintillation source, or the like.

### DETAILED DESCRIPTION OF THE INVENTION

The present invention in particular relies on annexin V (referred to herein generally as annexin) as the agent which localizes at a lesion or other target site within a blood vessel or other body lumen. Annexin V is a human protein (36 kD) of 319 amino acids. Annexin V binds with a high affinity to the phosphatidylserine moiety which is exposed on activated platelets present during thrombus formation within the vasculature. The use of technetium 99m-labeled annexin V for intra-arterial thrombus detection has been suggested in Stratton et al. (1995) supra. While the present invention will find particular use in the diagnosis and treatment of diseased lesions within the vasculature, most particularly in the diagnosis of coronary artery disease in the coronary vasculature, it will also be useful in a wide variety of other circumstances where uptake of a labeled substance can be related to diagnosis of a disease or other evaluation of a body lumen. For example, by introducing labeled annexin, various conditions related to excessive cellular proliferation can be assessed and monitored. For example, the presence or prognosis of various luminal cancers can be determined, such as cancer of the urinary bladder, colon cancer, esophageal cancer, prostate cancer (as well as benign prostate hyperplasia), lung cancer and other bronchial lesions, and the like, can be made.

The detection of the labeled annexin marker in situ within a body lumen has a number of significant advantages. Such in situ detection allows the detection of labels, such as visible light, fluorescence, luminescence, and the like, which cannot be deleted externally. With tissue-penetrating labels, such as radioisotopic radiation, in situ detection is much more sensitive than external detection. This is particularly the case when lower energy (short-path length) radiation sources are used, such as beta (β) radiation, conversion electrons, and the like. Detection of lower energy radiation reduces the background which is observed when the tracer concentrates in an adjacent organ or tissue, and is usually not feasible with external detection which, for example, relies on the introduction gamma (γ) radiation-emitting labels and the use of gamma (γ) cameras. The present invention, however, is not limited to the use of beta (β) radiation, conversion electrons, and other short path length radiation, but instead may find use with all types of ionizing radiation under appropriate circumstances.

In situ detection also improves detection of both the position and distribution of labeled immobilized within the body lumen. It will be appreciated that the detectors can be configured and/or repositioned so that immobilized radiation and other labels can be determined with an accuracy of less than 5 mm, usually less than 3 mm, preferably less than 2 mm, and often less than 1 mm, along the axis of the body lumen. The ability to accurately locate a target site, such as a region of unstable plaque, a region of proliferating cells, or the like, can greatly facilitate subsequent treatment.

The labeled annexin marker will comprise at least two components, i.e., a detectable label and annexin which acts as a binding substance. The detectable label can be any natural or synthetic material which is capable of in situ detection using an intravascular catheter or other intraluminal detector. Particularly suitable are radiolabels comprising radionuclides which emit beta (β) radiation, conversion electrons, and/or gamma (γ) radiation. Presently preferred are radiolabels which emit primarily beta (β) radiation or conversion electrons which have a relatively short path length and permit more precise localization of the target site or material. By using detector(s) capable of quantifying both beta (β) and gamma (γ) radiation, it will be possible to gauge how close the detector is to the label based on the observed ratio of beta (β)/gamma (γ) radiation and the known emission characteristics of the label. That is, the relative decline in observed beta (β) radiation will include that the detector is further from the label.

In addition to radiolabels, the present invention can employ other visible markers including fluorescent labels, such as fluorescein, Texas Red, phycocyanin dyes, arylsulfonate cyanine dyes, and the like; chemiluminescent labels, and/or bioluminescent labels. The present invention can also employ passive labels which respond to interrogation in various ways. For example, the labels may comprise paramagnetic or superparamagnetic materials which are detected based on magnetic resonance. Alternatively, the labels may be acoustically reflective or absorptive, allowing detection by ultrasonic reflection. Further, the labels could be absorptive or reflective to infrared radiation, allowing detection by optical coherence tomography.

The labels will typically be bound, covalently or non-covalently, to the annexin binding substance. Specific labeled annexin substances and methods for their production are taught, for example, in Stratton et al (1995) supra as well as U.S. Pat. Nos. 6,171,577 and 5,968,477, the full disclosures of which are incorporated herein by reference.

In addition to the labeled annexin substances described above, the methods of the present invention may also use a second binding substance (other than annexin) bound to a detectable label. Such additional binding substances can be virtually any material which becomes incorporated into and/or bound to a desired intraluminal target site. Thus, in the case of intravascular detection and labeling of atherosclerotic lesions, the second binding substance may be a natural substance which becomes incorporated into the lesions, such as low-density lipoproteins or components thereof. In the case of excessive self-proliferation, the second binding substances can be a variety of cellular precursors, including proteins, nucleic acids, and the like. In addition to natural materials which become incorporated into a growing or proliferating target site, the second binding substances can be prepared or synthesized for specific binding to a target site at the target location. For example, antibodies can be prepared to a wide variety of vascular and non-vascular target sites. Additionally, in some cases, natural receptors and/or ligands will be available for particular target sites. For example, monocyte chemoattractant peptide 1 (MCP1) localizes on receptors upregulated by the macrophages in plaque. Other target substance in plaque include lectins whose receptors are upregulated on endothelial cells that overly the plaque. Antibodies such as Z2D3 (Khaw et al., Carrio et al., Narula et al.) localize on proliferating smooth muscle in the plaque. Another potential agent is fluorodeoxyglucose labeled with fluorine-18. This agent emits positions and is utilized as an energy substrate by macrophages and monocytes, and it has shown enhanced localization in experimental atherosclerosis models.

The label and annexin or second binding substance may be bound to each other in any conventional manner. Most commonly, moieties on the label and/or the binding substance will be derivitized to permit covalent attachment to the annexin or second binding substance. Covalent attachment will usually be direct, but in some cases may employ a linking member. Non-covalent attachment can employ a variety of non-covalent linkers, such as biotin, avidin, intermediate antibodies, receptors, ligands, and the like. A variety of suitable binding techniques are described in a review article in Nature Biotechnology (1999) Vol. 17, pages 849 and 850, the full disclosure of which is incorporated by reference.

A variety of suitable labeled markers have been proposed in the medical and scientific literature. See, for example, U.S. Pat. Nos. 4,647,445; 4,660,563; 4,937,067; 4,877,599; 5,510,466; 5,711,931; 5,726,153; and WO 89/10760. Each of these patent references is hereby incorporated in its entirety by reference.

An important aspect of the present invention is the ability to detect and/or image the label in situ after the label has localized in the blood vessel wall or other body lumen. Because the label binds to specific target materials within the body lumen, the pattern in which the label has localized will correspond to the pattern of the target material in the body lumen. Such separate detection may be performed simultaneously, sequentially, or in some combination thereof. For example, the annexin as well as certain second labeled binding substances, such as low-density lipoproteins, or a component thereof, will bind to atherosclerotic plaque which is actively growing or accumulating and therefore at risk of being unstable. The pattern of label(s) will thus correspond to the pattern of unstable plaque within the patient's vasculature.

Detection of the label and its pattern within the body lumen will be performed using an intraluminal detector, usually a detector capable of detecting ionizing radiation from a radioisotopic label within a particular distance of the label, as discussed in more detail below. The detector and catheter can be introduced into the body lumen by a variety of conventional techniques. For intravascular detectors the preferred techniques will be percutaneous, e.g., using a needle and sheath for introduction of a guidewire in a Seldinger access technique. Alternatively, surgical cutdowns can be used for accessing blood vessels, and a variety of other surgical and minimally invasive techniques can be used for introducing intraluminal detectors into other body lumens.

The nature of the label and characteristics of the detector will be selected so that an emitted signal from the label will be visible or detectable only within a particular distance of a detecting surface or element of the detector usually within 5 mm, preferably within 3 mm, and sometimes within 1 mm. That is, the detector will only have a limited range for viewing localized label so that background from label located remotely from the detector will not be detected. In this way, accurate positional detection of the label can be achieved. In a presently preferred embodiment, the label will emit beta (β) radiation or conversion electrons or low energy x-rays which have a very short path length. The sensitivity of the detector will then be selected so that the beta (β) radiation will be visible only over a very short distance, typically less than 3 mm, preferably less than 1 mm. Moreover, the detector may be configured so that its detector surface(s) or element(s) will be engaged directly against the wall of the blood vessel or other body lumen to enhance detection of the charged particle radiation.

In a particular aspect of the present invention, detection of the label will be performed over a minimum length of the body lumen in order to characterize variations in the luminal lesion over that length with the ability to distinguish lesions present at intervals of 3 mm. For example, in blood vessels, the present invention will usually be used to image over a vascular length of at least 30 mm, preferably at least 40 mm, and more preferably at least 50 mm. Such detection may be achieved by scanning a detector over the length within the blood vessel or other body lumen. Preferably, however, the detector can remain stationary within the lumen and have spatial resolution over the preferred minimum length set forth above without movement of the detector itself.

In addition to the minimum detection lengths set forth above, the detectors will preferably be isotropic over at least their circumference or periphery. Regardless of whether the detector is scanned or held stationary during detection, it will normally be preferred that detection of label over the entire circumference or periphery of the body lumen be performed. In other cases, however, it might be desired to perform a directional scan i.e., one where a particular radial sector of the body lumen wall is observed.

In some cases, it may be preferred to employ two or more labels (which may be an annexin only or on second binding substances) and to separately detect those labels in order to determine the special distribution of more than one material in the body lumen. For example, in addition to annexin which localized on activated platelets, plaques at different phases of development have varying degrees of smooth muscle proliferation (detectable with Z2D3 antibody localization), varying degrees of macrophage infiltration (detectable with MCP1), varying levels of macrophage metabolism (detectable with the metabolic substrate FDG), and varying degrees of metalloproteinase activity (detectable with labeled antibodies specific for the metalloproteinase may be detected). Two or more parameters could be evaluated simultaneously if the radiopharmaceuticals carry radiolabels with substantially different energies or if one radionuclide has a substantially shorter half life than the other(s). Alternatively, labels having different natures, e.g., light emission, fluorescence emission, and/or radioisotopic radiation could be employed and detected simultaneous with minimum interference.

Detection of the localized annexin marker (either alone or in combination with a second or further marker) can provide useful information regarding a lesion or other structural condition of the body lumen. As described above, the present invention will permit determination of the axial and circumferential distribution of the target material within the body lumen. In the case of atherosclerotic lesions in a blood vessel, this information is particularly suitable for assessing the need for treatment as well as planning particular treatment modalities. In particular, the present inventor would allow the identification of relatively small lesions, e.g., with luminal blockage below 50%, which nonetheless are unstable and require immediate intervention. Conversely, larger lesions (above 50% occlusion) which are stable and less in need of immediate intervention can also be identified.

While the present invention is directed at intraluminal detection of marker(s), it may find use in combination with external detection of the same or other markers and/or external detection and imaging of the catheter which is being used for the intraluminal detection. External detection of immobilized markers may be useful for pre-positioning of the intraluminal detection catheter and/or for comparing information from different markers and targets (where the different markers may be bound to different binding substances having different specificities). External detection of the catheter will allow mapping of the vasculature or other luminal system. The position of the catheter can be detected fluoroscopically, by MRI, or otherwise, and the position of the internally detected lesions be noted on the external image or map which is created.

The methods of the present invention rely on the use of radiation detection devices comprising an elongate body, typically a catheter, and a radiation detector disposed on the elongate body. The catheter or other elongate body is configured to access the interior of a target body lumen, such as a blood vessel, a ureter, a urethra, an esophagus, a cervix, a uterus, a bladder, or the like. The radiation detector is capable of sensing radiation emitted into the body lumen and which is incident along the elongate body. In a first particular embodiment, the radiation detector will be capable of sensing radiation over a length of at least 3 cm, preferably at least 4 cm, and more preferably at least 5 cm. Optionally, the radiation detector will be capable of sensing radiation isotropically preferably being equally sensitive in all radial directions over the circumference of the elongate body.

In a second specific embodiment, the radiation detectors of the present invention will be capable of distinguishing radiation from at least two different radioactive labels with energies that differ by a threshold level.

In a third specific embodiment, the radiation detectors of the present invention will be capable of being axially translated within the body to sense radiation incident along the body over a length of at least 3 cm, preferably at least 4 cm, and more preferably at least 5 cm. Usually, such devices will comprise a catheter having an outside body which can remain stationary within a blood vessel and an internal detector which can be axially translated within the stationary body. Alternatively, the entire catheter may be translated within the lumen to cover the desired length.

Optionally, the catheters may comprise two or more different detection systems. Thus, in addition to the label detection system, the catheters might further indicate optical, ultrasonic, OCT, MR or other imaging systems. This will allow image information from the catheter to be “registered” or coordinated with the lesion characteristics also detected by the catheter. In some instances, it might be useful to provide for catheter-based excitation of a first or second label which has been immobilized at a target site.

As generally described to this point, the labeled annexin compositions are disclosed in prior pending U.S. Application No. 60/270,884 (Attorney Docket No. 20039-001500). For use in the present application, the compositions will usually comprise an additional effector molecule, as described above. The effector molecule can be bound to the annexin/labeled marker by any conventional technique, such as covalent binding. Binding of the three components or moieties of the compositions of the present invention will be achieved in such a way that the binding or other activity of the moiety is not significantly reduced so that the use of the compositions as described herein would fail.

While the above is a complete description of the preferred embodiments of the invention, various alternatives, modifications, and equivalents may be used. Therefore, the above description should not be taken as limiting the scope of the invention which is defined by the appended claims.

What is claimed is:

1. Compositions for detecting and treating vulnerable plaque, said compositions comprising:
a binding molecule which specifically binds to markers on stressed or apoptotic cells which are characteristic of vulnerable plaque;
a targeting molecule coupled to the binding molecule which permits localization of the composition when intravascularly bound to vulnerable plaque; and
an effector molecule coupled to the binding molecule which selectively kills or inhibits the stressed or apoptotic cells.

2. Compositions as in claim 1, wherein the binding molecule comprises an annexin.

3. Compositions as in claims 1 and 2, wherein the targeting molecule composes a radiolabel such as technetium-99m.

4. Compositions as in claims 1-3, wherein the effector molecule comprises a photodynamic agent such as a porphyrin.

5. Compositions for detecting and treating vulnerable plaque, said compositions comprising:
an annexin; and
a targeting molecule coupled to the annexin which permits localization of the composition when intravascularly bound to vulnerable plaque.

6. Compositions as in claim 5 wherein the annexin is annexin V.

7. Compositions as in claim 5 or 6, wherein the targeting molecule comprises a radiolabel such as technetium-99m.

8. A method for detecting and treating vulnerable plaque, said method comprising:
administering a composition to a patient suspected of having vulnerable plaque, said composition being capable of specifically binding to the vulnerable plaque, being localized when bound, and killing or inhibiting the apoptotic or stressed cells characteristic of vulnerable plaque;
determining whether the composition has localized within the vasculature, and
activating the composition to kill or inhibit the apoptotic or stressed cells the composition has localized.

9. A method as in claim 8, wherein the composition further comprises a photodynamic agent such as a porphyrin.

10. A method as in claim 8, wherein activating comprises photodynamic activation.
